SEQUENCE LISTING (4 sequences)

SEQ ID NO: 1 - DNA, 2211 bp, Artificial
  CD4 beta lactamase fusion
aagcttatgaaccggggagtcccttttaggcacttgcttctggtgctgcaactggcgctcctcccagcagccactcagggaatggacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggggcggcggcggctctggcggcggcggctctaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaatgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttgactcgag

SEQ ID NO: 2 - DNA, 2136 bp, Artificial
  CD4 Green Fluorescent Protein fusion
aagcttatgaaccggggagtcccttttaggcacttgcttctggtgctgcaactggcgctcctcccagcagccactcagggaatggtgagcaagggcgaggagctgttcaccggggtggtgcccatcctggtcgagctggacggcgacgtgaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggcggcggctctggcggcggcggctctaagaaagtggtgctgggcaaaaaaggggatacagtggaactgacctgtacagcttcccagaagaagagcatacaattccactggaaaaactccaaccagataaagattctgggaaatcagggctccttcttaactaaaggtccatccaagctgaatgatcgcgctgactcaagaagaagcctttgggaccaaggaaacttccccctgatcatcaagaatcttaagatagaagactcagatacttacatctgtgaagtggaggaccagaaggaggaggtgcaattgctagtgttcggattgactgccaactctgacacccacctgcttcaggggcagagcctgaccctgaccttggagagcccccctggtagtagcccctcagtgcaatgtaggagtccaaggggtaaaaacatacagggggggaagaccctctccgtgtctcagctggagctccaggatagtggcacctggacatgcactgtcttgcagaaccagaagaaggtggagttcaaaatagacatcgtggtgctagctttccagaaggcctccagcatagtctataagaaagagggggaacaggtggagttctccttcccactcgcctttacagttgaaaagctgacgggcagtggcgagctgtggtggcaggcggagagggcttcctcctccaagtcttggatcacctttgacctgaagaacaaggaagtgtctgtaaaatgggttacccaggaccctaagctccagatgggcaagaagctcccgctccacctcaccctgccccaggccttgcctcagtatgctggctctggaaacctcaccctggcccttgaagcgaaaacaggaaagttgcatcaggaagtgaacctggtggtgatgagagccactcagctccagaaaaatttgacctgtgaggtgtggggacccacctcccctaagctgatgctgagcttgaaactggagaacaaggaggcaaaggtctcgaagcgggagaaggcggtgtgggtgctgaaccctgaggcggggatgtggcagtgtctgctgagtgactcgggacaggtcctgctggaatccaacatcaaggttctgcccacatggtccaccccggtgcagccaatggccctgattgtgctggggggcgtcgccggcctcctgcttttcattgggctaggcatcttcttctgtgtcaggtgccggcaccgaaggcgccaagcagagcggatgtctcagatcaagagactcctcagtgagaagaagacctgccagtgccctcaccggtttcagaagacatgtagccccatttgactcgag

SEQ ID NO: 3 - DNA, 8304 bp, Artificial
  tissue plasminogen activator fusion
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattagatctatggagacagacacactcctgctatgggtactgctgctctgggttccaggttccactggtgactatccatatgatgttccagattatgctggggcccagccggccatggtttcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacaccaaggttaccaactacctagactggattcgtgacaacatgcgaccgccgcggctgcaggtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttaggcggccgctcgagatcagcctcgactgtgccttctagttgccagccatctgttgtttgcccctcccccgtgccttccttgaccctggaaggtgccactcccactgtcctttcctaataaaatgttaacgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcatgggtaacagtttcttgaagttggagaacaacattctgagggtaggagtcgaatattaagtaatcctgactcaattagccactgttttgaatccacatactccaatactcctgaaatagttcattatggacagcgcagaagagctggggagaattaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggcgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggcgattagtccaatttgttaaagacaggatatcagtggtccaggctctagttttgactcaacaatatcaccagctgaagcctatagagtacgagccatagataaaataaaagattttatttagtctccagaaaaaggggggaa

SEQ ID NO: 4 - DNA, 6501 bp, Artificial
  pSIREN retroviral vector encoding shRNA
gaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagcccaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcggcgtctcgagatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagggtcgaccctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtagatccggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgctctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattgaagatctgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaggatccggccacactccttgccctttttcaagagaaaagggcaaggagtgtggccttttttg

(19) United States
(12) Patent Application Publication
(10) Pub. No.: US 2008/0233558 A1
(43) Pub. Date: Sep. 25, 2008

(54) Inhibitors of viral entry screening method

(75) Inventor: Christoph Merten, Bottrop (DE)

Correspondence Address: EDWARDS ANGELL PALMER & DODGE LLP, P.O. BOX 55874, BOSTON, MA, 02205, US

(73) Assignee: Medical Research Council, London (GB)

(21) Appl. No.: 11/888,801

(22) Filed: Aug. 1, 2007

Related U.S. Application Data
(63) Continuation of application No. PCT/GB06/00316, filed on Jan. 31, 2006.
(60) Provisional application No. 60/648,827, filed on Feb. 1, 2005.

(30) Foreign Application Priority Data
Jan. 31, 2006 (GB) PCTGB2006000316

(51) Int. Cl. C12Q 1/70; C12Q 1/68; C07H 21/00; C07K 14/00

(52) U.S. Cl. 435/5; 435/6; 536/23.1; 530/350

(57) ABSTRACT

In one aspect the invention relates to a method for identifying inhibitors or viral entry comprising providing an indicator cell wherein said cell expresses a reporter gene and wherein said cell is capable of supporting entry by an effector particle, providing a candidate inhibitor of viral entry, co-compartmentalizing said candidate inhibitor and said indicator cell, contacting said indicator cell with an effector particle, incubating to allow any effector particle entry to take place, and assaying said indicator cell for reporter gene activity, wherein detection of reporter gene activity identifies the candidate inhibitor as an inhibitor for viral entry. Preferably the effector particle is HIV, preferably the reporter gene is a CD4-β-lactamase fusion or a tPA fusion, preferably the reporter gene activity is assayed by cleavage of an inert substrate into a fluorescent product.

### RELATED APPLICATIONS

This is a continuation patent application that claims priority to PCT patent application number PCT/GB2006/000316, filed on Jan. 31, 2006, which claims the benefit of Provisional patent application No. 60/648,827, filed on Feb. 1, 2005, the entirety of which are herein incorporated by reference.

### FIELD OF THE INVENTION

The invention relates to assays for studying viral infection and/or effector particle entry into cells. Typical effector particles would be pseudo-typed viral particles, or wild-type viral particles. Furthermore the invention relates to selection of sells resistant to infection and to identification of inhibitors of infection/entry.

### BACKGROUND TO THE INVENTION

Viral infections are a continuing threat to health throughout the world, in particular, human health. The number of casualties of human immunodeficiency virus (HIV) alone was three million in 2003, and the number of casualties for hepatitis exceeded one million. Furthermore, a new viral species such as the avian flu virus (often referred to as “bird flu”) continue to be identified and can become extremely dangerous for other species such as humans.

There is a clear need for tools for the study of these viruses, and in particular for the assay of potential modulators of viral entry and infection.

Existing viral infection assays are based on the expression of a reporter gene upon viral infection. For example, so-called LTR-driven reporter genes have been established to monitor HIV infections. In such a prior art system, a gene encoding a fluorophore such as green fluorescent protein (GFP) is arranged to be expressed in a cell upon infection with HIV. The assay read-out is fluorescence of said GFP. One of the problems with this system is that positive signal is coupled to infection and not to inhibition.

Thus, known assays for the inhibition of viral infections couple a positive readout (e.g. a Fluorescence signal) to the infection itself and not to its inhibition. These systems are based On the expression of a reporter gene (e.g. gfp) (within the host cell upon viral cell entry. When screening for potential inhibitors of viral infection, viral particles and host cells are incubated in presence of drug candidate(s). Subsequently, the reporter gene expression (e.g. fluorescence) is determined. A decreased signal in a given sample (in comparison to the control sample without any drug) should therefore result from a potent inhibitor of viral cell entry. However, a drug candidate that inhibits the reporter gene expression (e.g. by killing the host cell) rather than viral cell entry will inevitably be selected as a false positive in prior art systems. Furthermore, adverse side effects of the drug candidate on the host cell cause similar problems.

Adelson et al (Antimicrob Agents and Chemotherapy 2003 pages 501-508) disclose the development of a virus cell based assay for studying novel compounds against HIV1. The systems disclosed in this publication involve using established replication deficient HIV based vectors. These vectors are equipped with reporter genes such as GFP. The assays are partly conducted in producer cell lines and partly conducted in packaging cell lines. The processing and life cycle of these viral vectors are monitored within these different cellular contexts. All of the reporting and readout of these assays is based on reporter genes such as GFP which are carried on the viral vectors. Compounds which switch off the reporter genes are considered interesting. Clearly, these assays are not capable of distinguishing between generally cytotoxic compounds and those which have a specific effect on the viral life cycle. The cell lines used in these methods do not express reporter genes.

U.S. Pat. No. 6,884,576 discloses methods of monitoring HIV drug resistance. This system is founded upon the use of recombinant cells comprising reporter gene whose expression is regulated by proteins specific to HIV viruses which are expressed by the genome of an HIV virus upon infection of the recombinant cell by that virus. Regulation of the expression of this reporter gene is discussed in column 9 of U.S. Pat. No. 6,884,576. It is explained there that the regulatory protein responsible for modulating the expression of the reporter gene may be an HIV transactivator, HIV accessory protein, HIV structural protein or HIV enzymatic protein. Examples of these different possibilities are given. Thus, U.S. Pat. No. 6,884,576 appears to be primarily concerned with utilising viral effects on particular promoters in order to operate the assays. Dong's system couples expression of the reporter to infection, thereby producing a positive readout when a virus infects the cell. The assays described by Dong require viral replication, so interference with any aspect of the viral life cycle may result in a positive signal in this system. Lastly, Dong's system cannot distinguish non-infection related events (such as loss of the viral receptor) and is thus prone to selection of false positives on this account.

Siegert et al. (2005 AIDS Res. and Therapy vol. 2 p. 7) disclose assessment of HIV-1 entry inhibitors using MLV/HIV-1 pseudotyped vectors. They disclose MLV particles pseudotyped with HIV-1 env protein and bearing a retroviral vector genome encoding green fluorescent protein (GFP). Again, this system is based on the principle that successful infection leads to expression of GFP from the incoming viral genome, so that inhibition of infection leads to lack of signal. This system suffers from the problem that inhibition of any aspect of the signalling system itself will cause a ‘false positive’ readout.

The present invention seeks to overcome problem(s) associated with the prior art.

### SUMMARY OF THE INVENTION

The present invention is based on the surprising finding that cellular downregulation of certain genes (e.g. viral receptors such as CD4), mediated by viral particle entry, can be used to create alternative assays.

An advantage of the present invention is that the systems couple inhibition of infection to a positive signal, rather than the prior art coupling of infection to a positive signal. This feature advantageously reduces the selection of false positive compounds, such as those compounds inhibiting the signal by some mechanism, but without having a specific inhibitory effect on infection/entry.

Thus the invention provides assay systems which generate a steady-state signal in the absence of effector particle infection (e.g. virus infection), but when infection takes place, downregulation of the signal elements occurs leading to loss of read-out. Thus, only those cells remaining uninfected continue to produce signal and thereby identify the presence of inhibitors of infection. This is in stark contrast to prior art systems where any input to the system which compromises the signal leads to a ‘positive’ result, whereas advantageously the present invention provides for a system where prevention or inhibition of infection itself leads to a sustained signal, reducing or even eliminating false positives from the assays.

In one aspect the invention relates to a method for identifying inhibitors of viral entry comprising providing an indicator cell wherein said cell expresses a reporter gene and wherein said cell is capable of supporting entry by an effector particle, providing a candidate inhibitor of viral entry, co-compartmentalising said candidate inhibitor and said indicator cell, contacting said indicator cell with an effector particle, incubating to allow any effector particle entry to take place, and assaying said indicator cell for reporter gene activity, wherein detection of reporter gene activity identifies the candidate inhibitor as an inhibitor of viral entry.

Co-compartmentalising preferably means that the elements are in the same aqueous phase such that they may contact one another. Co-compartmentalising may mean that the elements are within the actual cell e.g. when the candidate inhibitor is expressed by the indicator cell it may be regarded as being ‘co-compartmentalised’ with that cell.

The candidate inhibitor may be any agent such as a chemical entity which it is desired to test. The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may be a polynucleotide molecule. The agent may be an antibody. The agent may be designed or obtained from a library of compounds, which may comprise peptides, as well as other compounds, such as small organic molecules. By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as using a peptide synthesiser or by recombinant techniques or combinations thereof). Typically, the agent will be an organic compound. Typically, the organic compound will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, alkyl groups, cyclic groups etc; substituent groups may be unbranched- or branched-chain. In addition to the possibility of the substituents being cyclic groups, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group.

Preferably the candidate inhibitor is a polypeptide. When the candidate inhibitor is a polymer such as a polynucleotide or a polypeptide, preferably the candidate inhibitor is supplied by production in the indicator cell. This may be by introduction of an expression library encoding candidate inhibitors such as a peptide library.

Capable of supporting entry means that the effector particle can breach the cell surface and deliver its nucleic acid to the inside of the cell in the usual manner, unless an inhibitor is present. To render a cell capable of supporting entry, the appropriate viral receptors/co-receptors may need to be supplied such as by transfection or transduction of constructs capable of directing their expression.

The cell expresses a reporter gene. This may be caused by transfection or transduction of the reporter gene, preferably transduction. Said transfection or transduction may be transient or stable, preferably stable. Most preferably the indicator cell expresses a reporter gene which has stably integrated into the genome of the cell. Preferably this has been accomplished before any contact with an effector particle.

The incubation step is to allow for any entry to take place, if it is possible. Clearly, when an inhibitor is present entry will not be possible i.e. it will be inhibited. The incubation should be of suitable duration that when no inhibitor is present normal entry occurs and the expected downregulation of the reporter gene takes place. The time required for this will vary depending on the cell, effector particle and/or reporter systems chosen. The precise time of incubation for a given system may be determined by conducting the assay without inhibitor over a time course and choosing the time at which entry has occurred and reporter gene expression has been shut down.

The reporter gene may be assayed by any suitable means, as described below.

The reporter gene may encode a fluorophore or a chromophore or other entity capable of direct detection.

Preferably the reporter gene encodes an enzyme or active fragment thereof capable of converting a fluorogenic or chromogenic substrate to a fluorophore or chromophore whose presence can be detected thereby. The enzyme may be an intracellular enzyme or may be displayed on the cell surface. Preferably the enzyme or fragment is displayed on the cell surface. Preferably this cell surface localisation may be achieved by fusion to a viral receptor or transmembrane domain, preferably a viral receptor. This preferred embodiment has the further advantage that presence of the enzyme or part thereof on the cell surface is an indicator that the viral receptor is correctly expressed and displayed on the cell surface, enabling easy internal validation of the assays.

Preferably the reporter gene product is directed to the cell surface. This may be by fusion to a cell surface protein such as CD4, or may be by incorporation (e.g. fusion) of a suitable signal sequence such as a transmembrane domain e.g. PDGFR-TM. Preferably that part of the reporter gene product which mediates detection is extracellular. This enables easy access to reagents/substrates used for detection without having to propel them across the cell membrane.

Preferably the reporter gene is tissue plasminogen activator (tPA) or β-lactamase.

Preferably the reporter gene is fused to a gene known to be downregulated upon entry of the effector particle (such as a virus particle). Preferably this fusion is fusion of the coding sequences such that a gene product comprising the reporter element and the element known to be downregulated upon entry of the effector particle is directed to be produced as a single (fused) polypeptide.

Preferably the gene known to be downregulated on viral entry is CD4.

Preferably the reporter gene comprises a CD4-reporter fusion such as a CD4-tPA fusion or a CD4-β lactamase fusion.

Preferably the reporter gene is under the control of a promoter which is known to be downregulated on viral entry.

Preferably the effector particle comprises nucleic acid encoding elements capable of inhibiting expression of the reporter gene.

Preferably the effector particle comprises nucleic acid encoding shRNA capable of inhibiting expression of the reporter gene.

Preferably the effector particle comprises a virus, preferably the virus is a recombinant virus, preferably the virus is a pseudotyped virus. In another embodiment, preferably the virus is a wild-type virus, which offers the advantage of providing an assay closer to the biological situation.

Preferably co-compartmentalisation is by forming one or more aqueous droplets comprising both the candidate inhibitor and the indicator cell. For some embodiments, co-compartmentalisation is preferably by forming one or more aqueous droplets comprising the candidate inhibitor and the indicator cell and the effector particle(s).

Preferably the aqueous droplets are part of a water-in-oil emulsion.

Preferably the aqueous droplets are part of a water-in-oil-in-water emulsion.

Preferably the candidate inhibitor is produced by the indicator cell. This may be due to transfection of a gene capable of directing expression of the candidate inhibitor. Transfection may be stable or transient, preferably stable.

Preferably the reporter gene encodes an enzyme, or an active fragment thereof, and detection of reporter gene activity comprises contacting said indicator cell with a substrate for said enzyme, incubating to allow said enzyme to act on said substrate, and detecting the presence of enzymatic product, presence of the product indicating reporter gene activity.

Detection may be by fluorescent resonance energy transfer (FRET), by change in fluorescence and/or absorbance, by abolition of fluorescence and/or absorbance or by generation/initiation of fluorescence and/or absorbance at the appropriate wavelengths. Preferably detection is by generation/initiation of fluorescence (or absorbance) wherein the substrate is non-fluorescent (or non-absorbent) but the cleaved product is fluorescent (or absorbent). In other words (or alternatively) detection may be by discernibly different fluorescence (or absorbance) spectra of substrate and product.

In another aspect, the invention provides a method as described above wherein detection of reporter gene activity comprises detection of reporter gene expression by contacting said indicator cell with an antibody capable of reacting with said reporter gene product; incubating to allow binding of said antibody to said reporter gene product; and detecting the presence of bound antibody on said indicator cell, presence of the antibody indicating reporter gene expression. When using antibody detection, the reporter gene may be advantageously adapted to include a peptide tag (such as the HA tag, myc tag, flag tag or any other suitable tag) to facilitate its detection. By ‘reacting with’ is meant binding, association or other interaction whereby the antibody becomes associated with the reporter gene product such that detection (or localisation) of the antibody can be taken to indicate presence (or location) of the reporter gene product.

Preferably the effector particle is HIV, preferably the reporter gene comprises a CD4-tPA fusion, preferably the reporter gene activity is assayed by cleavage of an inert substrate into a fluorescent product.

### DETAILED DESCRIPTION OF THE INVENTION

The present invention provides a universal, rapid and sensitive assay to screen and select compounds (e.g. small molecules, peptides, proteins, antibodies) for their ability to inhibit viral infections. It is based on the expression of a reporter gene in the target cells in a way that results in downregulation upon infection with an effector particle such as a particular virus. Thus, the approach described herein couples a positive signal with the inhibition of an infection rather than, as in already existing assays, with the infection itself.

In contrast to prior art techniques, the positive signal viral inhibition assays (PSVIA) of the present invention represent a system that couples a positive readout signal to the inhibition of viral infection. Consequently, the probability of selection of false positives is significantly decreased and the system favours drug candidates that do not harm the host cells. In addition, the direct coupling of a positive signal to the desired property is highly advantageous for directed evolution strategies and high throughput screening (HTS).

The invention makes use of genetically engineered host cells (indicator cells) that express (preferably constitutively) a membrane-bound affinity tag and/or reporter enzyme. Consequently, these cells can be stained with antibodies and/or assayed for conversion of a non-fluorogenic substrate into a fluorogenic product. To assay the inhibition of viral cell entry, the indicator cells can be incubated with effector particles (e.g. viral particles). These may transduce gene(s) downregulating the reporter gene expression (e.g. based on sh- or antisense RNA, transcriptional repressors or suicide genes), or may downregulate the expression by the mechanism of entry itself. Thus, effector particle entry results in a decreased reporter gene signal, whereas non-transduced cells show the maximum signal intensity.

In a preferred embodiment, the current system is based on indicator cells expressing a membrane-bound and HA-tagged form of the human tissue plasminogen activator (tPA-HA). This enzyme converts plasminogen into plasmin which then converts a non-fluorogenic substrate into a fluorogenic product. As effector particles, MLV(VSV-G Env) pseudotyped particles are preferred. These particles have packaged a vector encoding shRNA against the tPA-HA. Upon cell entry of the effector particles, the shRNA is expressed in the indicator cells resulting in downregulation of the tPA-HA.

Clearly in some embodiments the detection of tPA may be by its direct action on a chromogenic or fluorogenic substrate, rather than its action on plasmin and the subsequent action of plasmin on a chromogenic or fluorogenic substrate.

It is an advantage of the invention that the assays have a decreased probability of selecting false positive inhibitors compared to prior art techniques.

The invention enables easy determination of optimal inhibitor concentrations. Compatibility with directed evolution approaches is provided by the assays disclosed herein.

The invention provides high flexibility, and allows selection of inhibitors of different viral species.

The invention has numerous safety features such as alleviating the need to work with wild-type virus, for example using pseudotyped particles. However, some embodiments involve the use of actual virus particles, which is advantageous in studying the behaviour of the most clinically relevant virus samples.

Since in preferred embodiments the invention can use non-replication competent retroviral pseudotyped particles instead of wild type virus, it offers further advantages over existing technology. Firstly, in this embodiment all work can be performed in containment level one laboratories since live virus is not required.

Furthermore, the modular system of pseudotyping allows selection of inhibitors of different viral species. For that purpose, only the applied envelope protein has to be exchanged. Since the tropism of a retroviral particle is determined by its envelope protein (Env), exchanging the VSV-G protein against envelope proteins of other viral species (e.g. HIV, HCV, Coronaviruses associated with SARS, influenza) results in cell-entry assays for a variety of viruses. The applied indicator cell line can advantageously be the same for different viral species, so long as the corresponding receptor(s) are expressed by that cell line (whether endogenously or by genetic modification of the cell line to provide receptor expression). Therefore, the assays of the invention can easily be modified for varied applications.

Definitions

As used herein, the term ‘indicator cells’ means any suitable cells which are capable of supporting reporter gene expression and are capable of supporting effector particle entry. When the effector particle is a virus, preferably the indicator cells are derived from the natural host species of said virus. Preferred indicator cells are 293 EBNA T cells or HEK293T cells; preferably indicator cells are derived from HEK293T cells.

As used herein, the term ‘effector particles’ means any particle capable of emulating infection, transduction or cell entry by a pathogen. The particle may be any particle useful in the simulation or emulation of infection by a pathogen. Most preferably the particles can carry a nucleic acid moiety and are capable of delivering this to the inside of an indicator cell, preferably by a mechanism similar to that of a pathogen such as a virus, preferably by a mechanism identical to that of a pathogen such as a virus. Preferably the particles are (or are derived from) viruses or virus lice particles, preferably gamma-retroviruses or lentiviruses, preferably lentiviruses; preferably recombinant viruses; more preferably recombinant viruses pseudotyped with heterologous envelope protein(s).

Recombinant effector particles may be employed such as pseudotyped particles comprising a nucleic acid capable of bringing about downregulation of the reporter gene (such as shRNA, transcription factors, antisense RNA or suicide genes; preferably shRNA) and displaying an envelope protein of a viral species of interest. Said envelope protein may be modified (e.g. C-terminally truncated) if desired. Pseudotyping and related techniques are well known in the art.

By using recombinant effector particles, the assays of the invention can easily be modified for different viral species. Simply by exchanging the viral envelope protein expressed in the packaging cells (and subsequently displayed on the particles), inhibitors against a variety of species can be selected. There is no need to alter the nature of the packaged nucleic acid element of the vector, nor to create a new reporter gene construct. Advantageously there is not even a requirement for species-specific indicator cells, as long as the corresponding viral receptors are expressed.

As used herein, the term ‘reporter genes’ has its normal meaning in the art, i.e. of a gene whose product can be readily detected, for example so as to derive information about the expression state of said gene. Typical reporter genes include fluorescent proteins or enzymes. A preferred reporter gene is β-lactamase (beta-lactamase) or tissue plasminogen activator (tPA) which are both well known in the art; preferably the reporter is tPA.

Preferably a reporter gene encodes a cell surface directed reporter gene product. This may be achieved by fusion with a cell surface receptor such as CD4 or may be achieved by fusion with a transmembrane domain such as PDGF-TM.

Preferably the reporter gene comprises a signal sequence directing trafficking of the gene product to the cellular membrane.

Preferably the reporter gene comprises a CD4-reporter fusion such as a CD4-tPA fusion or a CD4-β lactamase fusion, or a PDGF-TM-reporter fusion such as a PDGF-TM-tPA fusion or a PDGF-TM-β lactamase fusion.

Preferably a reporter gene comprises an antibody recognition sequence such as a HA tag, a myc tag, a flag tag or other epitope, preferably a HA tag.

Preferably a reporter gene comprises a signal sequence that ensures intracellular routing of proteins to the endoplasmic reticulum. Preferably this is an IG-K chain sequence. This is a basic requirement for proteins with extracellular domains (e.g. membrane-bound or released proteins). Any sequence that ensures routing to the extracellular side of the cell membrane could be used in place of IG-K.

As used herein, the term ‘reporter fusions’ refers to a reporter gene fused to another gene. This may involve fusion only of the coding sequence of the reporter gene to part or all of the coding sequence of another gene, whereby the whole of the fused coding sequence remains under the control of the other gene's control elements such as promoters, enhancers and the like. This simply results in production of a reporter fusion protein as is well known in the art. A preferred reporter fusion of the present invention is the fusion of β-lactamase of tPA to a cellular transmembrane domain (platelet derived growth factor receptor transmembrane domain; PDGFR-TM) or CD4 or as described in more detail below.

Applications

The invention finds application in many areas including high-throughput screens and directed evolution techniques, since it drastically reduces the selection of false positive compounds (compounds that bypass the selection criteria of a given infection assay but do not specifically inhibit infection). Furthermore, the present invention can advantageously be applied to different types, species or clades of virus. A further advantage is that pseudotyped particles can be used in the methods of the invention and therefore use of live or intact virus is advantageously avoided. This has another benefit in that high containment level work can be reduced or eliminated from the procedures, which improves safety and reduces the cost and administrative burden of the processes according to the present invention. Further applications and benefits are described herein.

The assays of the invention allow screening of drug candidates for inhibiting viral cell-entry and/or reverse transcription and/or integration into the host cell genome.

In particular, the invention finds application in the screening of small molecules within microtitre plates or microfluidic devices (emulsions), and screening genetically-encoded libraries of peptides, shRNAs or antibodies using FACS. This application advantageously allows new drugs and also new drug targets to be identified.

Furthermore the invention may be used to detect virus or infectivity in a sample. In this embodiment, indicator cells according to the present invention would be contacted with a sample thought to comprise the virus of interest. The reporter gene in the indicator cells will remain ‘on’ (i.e. giving continuous readout) in the absence of infection, but would be shut off (ie. signal lost) upon infection. Thus, if, following contact with the sample, the signal is lost then it would indicate that the sample is likely to have comprised the virus of interest.

Assays of the Invention

The present invention is based on genetically modified target cells (indicator cells which may comprise a stable cell line) expressing the viral receptor(s) of interest, together with any co-receptors which might be required for infection or entry. These cells are genetically modified in the sense that they express a reporter gene, such as an affinity tag, a fluorogenic protein or an enzyme able to convert substrates into fluorogenic, chromogenic or luminometric products. Coupling this type of reporter signal to an inhibition of viral infection is accomplished by arranging the expression of the reporter gene to be strongly decreased (downregulated) upon infection with the virus of interest. In principle, this can be ensured by any suitable means, but especially preferred are:

A) The reporter gene product itself is fused to a cellular protein which, upon infection with the virus of interest is itself downregulated. For example, the reporter gene product can be fused to the corresponding viral receptor, which in many cases is downregulated upon infection.

A specific example of such a system is the downregulation of the CD4 receptor upon HIV infection. In this scenario, the reporter gene is fused to CD4, whose expression is strongly decreased upon co-expression of HIV genes such as nef, env and vpu.

B) Effector particles are used which have packaged a nucleic acid encoding a gene product that interferes with the expression of the reporter gene. An example of such an effector particle is a recombinant viral particle. In particular, viral particles that have packaged a vector encoding short hairpin RNA (shRNA), antisense RNA or other transcriptional, translational and/or posttranslational repressors or suicide gene(s) can be used in the assay.

A specific example of such a system is the downregulation of cell surface-displayed tPA upon cell entry of MLV-derived recombinant pseudotyped particles which transduce a vector encoding shRNA against the tPA reporter gene (in this example the shRNA is targeted to the PDGFR-TM domain of the reporter gene).

Thus, the present invention provides a strategy to generate modular recombinant viral particles (recombinant effector particles) allowing to screen for inhibitors of completely different viral species. For this purpose, gamma retrovirus e.g. murine leukaemia virus-derived (MLV-derived) or lentiviral (e.g. HIV-derived) particles may be generated which have packaged a vector encoding shRNA targeted against the reporter gene in the indicator cell line and can functionally incorporate or display a variety of different envelope proteins on their surface. The resulting pseudotype particles thus show the host range tropism that is mediated by the corresponding envelope protein and can be used instead of wildtype viruses within the inhibition assay. This not only has strong safety benefits, but also advantageously broadens the application range of the invention.

Thus in one aspect a compound library may be screened for the ability to inhibit the infection of CD4-positive cells with the human immunodeficiency virus (HIV). An appropriate indicator cell line is generated that stably expresses a reporter gene fused to the CD4-receptor (the wildtype CD4 receptor can be expressed additionally, if the fusion protein does not mediate cell-entry of HIV particles—this can be easily determined by the person skilled in the art) and one or more of the required coreceptors (such as CXCR4, CCR5, etc.). These indicator cells are seeded in microtiter plates and incubated with HIV-1 particles (ie. effector particles) in presence of different compounds in each well. Upon infection, the reporter-CD4 fusion protein is downregulated due to the expression of the viral genes env, vpu and nef. Consequently, only cells that have not been infected with HIV will express the reporter gene. Thus, wells that exhibit a positive reporter signal contain compounds that inhibit HIV infection. Variations and modifications of these assays will be apparent from the relevant sections of the description which explain individual parts of the assay in more detail.

The invention may be applied to any suitable viral system selected for study. Particularly preferred are HIV (preferably with receptor: CD4 co-receptors: CXCR4, CCR5); Hepatitis C (HCV), Influenza and related species such as bird flu (cell entry via sialic acid receptors), or coronaviruses (cell entry via coronavirus receptors/aminopeptidases such as CEA family).

Assay Formats

Microfluidic handling techniques, emulsion based droplet compartmentalisation and microtitre plate wells (such as 12, 24 or 96-well format) are all useful formats for the assays of the present invention. These techniques are well known in the art. In particular, reference is made to WO99/02671 and WO00/40712 which both describe optical sorting methods of application to the methods described herein. The way in which the readout is collected and the optimal assay formats depend upon operator preferences. Factors to be taken into account may include the number of samples to be processed. For example, if sample numbers are small, it may be convenient to process them manually in a microtitre plate with manual pipetting; in this embodiment ‘co-compartmentalisation’ may refer to the elements being placed into the same microtitre well. However, where sample numbers are large, it may be more convenient to use an automated or semi-automated processing apparatus to conduct the screening and selection. These choices are well within the ordinary skill of the person working the invention.

ReportersSubstrates/Readout

The reporter may be detected directly (e.g. by antibody based detection) or indirectly (e.g. by assay of reporter activity). Direct detection of reporter gene activity may be based on the gene activity such as detection of transcription, translation or direct detection of the gene product. Indirect detection principally refers to assaying for activity of the gene product such as an enzymatic activity, e.g. by supplying a substrate and monitoring cleavage of same or by some similar technique.

In choosing a reporter enzyme, preferably it should mediate a rapid turnover of substrate (ie. have high Kcat/Km). Preferably is should be an enzyme for which fluorogenic and/or chromogenic substrate(s) are available.

Preferably the reporter enzyme or fragment thereof is displayed on the cell surface. Preferably the reporter gene comprises a surface targeting element such as a transmembrane domain to achieve cell surface localization of the reporter enzyme or fragment thereof. Preferred cell surface targeting element is a single-spanning membrane protein, or a single spanning domain from a multiple membrane-spanning protein. For example, the reporter gene could be fused to the SU domain of retroviral env protein(s), preferably N-terminally fused thereto. Especially preferred cell surface targeting agents are fusion to CD4 receptor, or fusion to the transmembrane domain of PDGF (PDGF-TM).

Expression of the reporter gene should preferably be driven by a strong promoter.

Preferably the reporter gene encodes an enzymatic activity, which activity is retained at the cell surface.

It will be noted that when the reporter enzyme activity is located at the cell surface, that the substrate for conversion to a chromogenic or fluorogenic product will also need to be available at the cell surface. Typically this is achieved by presenting the substrate extracellularly so that it will be able to be acted upon by the cell surface localized reporter enzyme activity. In these embodiments, it will be apparent that droplet co-compartmentalisation is advantageous in that it allows a pool of cleaved substrate to be detected in the extracellular part of the droplet and thereby associates that with the cell in the droplet. Thus, droplet format is advantageously used when selecting cells on the basis of extracellular readout. Alternatively the reporter gene product itself call be tagged, for example by reaction with an anti-reporter antibody. This advantageously allows individual cells to be selected without having to perform droplet co-compartmentalisation. The skilled worker may easily choose the format which best suits their application of the invention.

As is described herein, it will be noted that some reporter genes may give readout via intermediate steps. For example, when the reporter is tPA, then the readout is preferably via the action of tPA on plasminogen; this creates plasmin; the plasmin acts on the substrate such as HDLVK-Amc and this creates a fluorogenic product. Thus, when using multi-step readouts such as this, then each of the necessary elements must be provided to the indicator cells. In the case of tPA readout, this may involve supplying both plasminogen as well as HDLVK-Amc to the indicator cells to allow the readout to be produced.

When the reporter gene is β-lactamase, preferably Fluorocillin™ Green 495/525 β-lactamase substrate (Molecular Probes) is the substrate.

Plasminogen may be obtained from Roche, Switzerland. The plasmin substrate HDLVK-Amc is preferably used and may be obtained from Bachem, USA (see examples). Alternatively, other plasmin substrates such as Rhodamine 110-bisCBZ-L-Phe-L-Arg from Molecular Probes, USA may be used.

Downregulation

It is a key feature of the present invention that there is a steady-state signal in the absence of infection (effector particle entry/transduction). This advantageously allows for a two-fold output; firstly that infection or effector particle entry has not occurred (or has been inhibited at a downstream point) and secondly that the cell remains alive (i.e. there is no, or only limited, cytotoxicity). In order to obtain this key advantage of the invention, it is necessary that infection or effector particle entry downregulates the reporter gene. This may be achieved via wholly recombinant means (e.g. expression of a shRNA against a recombinant reporter gene) or preferably may be via a naturally occurring downregulation of a recombinant reporter gene (e.g. downregulation of a receptor to which the reporter gene has been fused), or may be by any other means known to those skilled in the art such as mediation of expression by a virus-specific protein. The important feature is that the downregulation of the reporter is coupled to infection so that resistant or inhibited cells (uninfected cells) continue to maintain the reporter readout.

In a broad sense, downregulation refers to downregulation of reporter gene activity or reporter gene product activity. In this sense it may refer to transcriptional and/or translational and/or post-translational deactivation as well as downregulation in the usual sense. For example, the downregulation leading to detectable loss of signal may be brought about by induction of misfolding, loss of a toxin inhibitor, inhibition of transport, failure or loss (removal) of posttranslational modification, introduction of destructive posttranslational modification (for example destructive to activity e.g. phosphorylation to inactive state, or destructive to existence e.g. ubiquitination for degradation), inhibition of activity, or other mechanism for knocking-down signal.

Downregulation of recombinant, cell surface-displayed reporter genes such as tPA may be employed in the present invention. tPA is an enzyme involved in fibrinolysis. It converts inactive plasminogen into active plasmin, which can then convert further substrates including artificial fluorogenic or chromogenic substrates. In a preferred embodiment, tPA is fused to a cellular transmembrane domain (preferably the platelet derived growth factor receptor transmembrane domain; PDGFR-TM) to achieve cell surface display. Consequently, pseudotyped particles that have packaged a vector encoding shRNA against the reporter gene will mediate downregulation of the reporter gene construct in the indicator cell upon effector particle entry (transduction). This mechanism is exploited in assays of the present invention, and is illustrated in FIG. 12.

Downregulation of reporter genes fused to viral receptors such as CD4 can also be employed in the present invention. In these embodiments, rather than fusing the reporter gene to non-viral transmembrane domains such as PDGFR-TM, the fusion is made with a viral receptor such as CD4. CD4 is a chemokine receptor which is used by HIV as a receptor to enter the cell. HIV proteins Nef, Vpu and Env mediate downregulation of CD4 after infection. This occurs by endocytosis/degradation of CD4. Downregulation of viral receptors after infection with the corresponding viral species is well characterised for HIV and for a wide variety of viruses. This mechanism is exploited in assays of the present invention, and is illustrated in the such as FIG. 1.

It is now discussed how the downregulation is incorporated into the assays of the invention.

The invention finds application in many different selection strategies. In one embodiment, the invention may be used in selection of genetically-encoded inhibitors such as antibodies or peptides inhibiting viral infection. FIG. 13 shows a diagram illustrating one such embodiment. In another embodiment, the invention may be used in screening of small molecules for activity in inhibiting viral infection. These applications benefit from the positive sorting signal which is advantageously provided by the present invention.

Inhibition allows positive selection, as illustrated in FIG. 13.

The principle of the inhibition assay is that inhibition of viral cell entry or early steps of the viral life cycle (such as reverse transcription or integration into the host cell genome) by a candidate inhibitor means that the reporter gene stays on the cell surface and maintains readout such as fluorescence due to product being produced from the substrate by the action of the cell surface reporter such as tPA (FIG. 13). Inhibition of these early steps of the viral life cycle is therapeutically superior to inhibition of later steps like viral assembly or budding since at that late stage the viral genome has already integrated into the host cell genome and is thus inevitably conserved. An alternative readout method is to stain the cells with antibodies raised against the reporter gene (FIG. 14).

The principle where the candidate substance is a non-potent inhibitor is shown in FIGS. 13B and 14B where there is no significant inhibition of infection (in this example the candidate inhibitor is a small molecule); this means that the effector particle can transduce the indicator target cell. When this happens, expression of the cell surface displayed reporter such as tPA is downregulated and because there is no reporter such as tPA on the surface then there is no fluorescence.

In another embodiment, the invention may be used to determine optimal concentrations of a given inhibitor. When effector particles and indicator cells are co-compartmentalized at different concentrations of the inhibitor, the resulting fluorescence will correlate with the number of transduction events (FIG. 15A). A major advantage of the invention over prior art assays is the fact that within the present assays adverse side effects of the inhibitor on the cells will cause a decreased fluorescence signal. This is due to the fact that only viable cells will express the reporter gene and thus generate a positive readout signal. In contrast, with prior art assays adverse side effects would be interpreted as potent inhibition since in this case successful inhibition is coupled to a negative readout signal (FIG. 15B).

Data are presented in FIGS. 16 to 19 which demonstrate the selection effects according to the present invention.

In a broad aspect the invention relates to techniques to select antibodies, peptides and small molecules inhibiting viral infection such as HIV, HCV or influenza infections. Preferably said techniques are compartmentalization-based.

Preferably the assay of the present invention uses one or more stable cell line(s) expressing indicator moiety fusion protein.

The present invention provides a novel assay which advantageously couples inhibition of viral cell-entry or later steps of the viral life cycle (such as reverse transcription and integration into the host cell genome) to a positive signal.

Advantageously signal to noise ratios enhance the selection procedures of the invention.

The invention finds application in many different selection strategies. In one embodiment, the invention may be used in selection of antibodies or peptides inhibiting HIV-infection. The selection procedure itself focuses on the enzymatic conversion of fluorogenic or chromogenic substrates or antibody staining of the reporter gene product as described above. Advantageously downregulation of the viral receptor (or receptor-reporter fusion as used herein) is a naturally occurring phenomenon which allows use of the virus itself as the effector particle. FIG. 1 shows such an embodiment.

In another embodiment, the invention may be used in screening of small molecules for activity in inhibiting HIV-infection. These applications require positive sorting signal which is advantageously provided by the present invention.

Expression allows positive selection, as illustrated in FIG. 4.

It is an advantage of the present invention that selection within droplets can be carried out (see FIG. 4).

The principle of the inhibition is that inhibition of infection by HIV such as by a potent inhibitor means that the reporter gene product (such as a CD4-β-lac fusion) stays on the cell surface and causes fluorescence due to product being produced from the substrate by the action of β-lac (e.g. see FIG. 5).

The principle where the candidate substance is a non-potent inhibitor is shown in FIG. 6 where there is no significant inhibition of infection (in FIG. 6 the candidate inhibitor is a small molecule); this means that the effector particle can effect cell entry as shown in FIG. 7 (the effector particle in this scenario is an HIV particle). When this happens, expression of the cell surface reporter (such as a CD4-β-lac fusion) is down regulated and because there is no β-lac on the surface then there is no fluorescence, allowing selection.

Data are presented in FIGS. 8 and 9 which demonstrate the selection effects according to the present invention.

In a broad aspect the invention relates to techniques to select antibodies, peptides and small molecules inhibiting viral infection such as HIV infection. Preferably said techniques are compartmentalisation-based.

It has been observed that the downregulation assay may not work well with GFP-CD4 fusions. Without wishing to be bound by theory, this may be due to fluorescence persisting after downregulation. Alternatively it may be due to degradation of this particular fusion being too slow. It is possible that the timepoints may be optimised to overcome the specific characteristics of the GFP-CD4 fusion, e.g. measurement at later timepoint (following day). Nevertheless, preferably the indicator moiety is not GFP. More preferably, when CD4 is fused to an indicator moiety, the indicator moiety is not GFP.

Preferably the assay of the present invention uses one or more stable cell line(s) expressing indicator moiety fusion protein.

The present invention provides a novel assay which advantageously couples HIV-inhibition to a positive signal.

Advantageously signal to noise ratios enhance the selection procedures of the invention.

Further Aspects

In another aspect the invention provides a method of screening compounds for their ability to inhibit viral infection comprising the steps of: (a) generating or using an indicator cell line that expresses a receptor for the virus of interest and a reporter protein fused to a cellular protein which is downregulated upon infection with the virus of interest, thus coupling a positive reporter signal to the inhibition of infection (b) screening compounds for their inhibitory effect on viral infection by incubating indicator cells with viral particles in presence of the compounds to be screened and subsequent determination of the reporter gene signal.

In another aspect the invention relates to a method of screening compounds for their ability to inhibit viral infection comprising the steps of: (a) generating or using recombinant effector particles that have packaged a nucleic acid encoding a gene product that interferes with the expression of a reporter gene in the indicator cell line, thus coupling a positive reporter signal to the inhibition of transduction (b) screening compounds for their inhibitory effect on viral cell-entry by incubating indicator cells with recombinant effector particles in presence of the compounds to be screened and subsequent determination of the reporter gene expression.

Preferably the indicator cells, the viral/effector particles and the compounds are incubated within microtiter plates.

Preferably the indicator cells, the viral/effector particles and the compounds are incubated within a microfluidics device.

Preferably the indicator cells, the viral/effector particles and the compounds are incubated within aqueous droplets of an emulsion.

Preferably the indicator cells, the viral/effector particles and the compounds are incubated within aqueous droplets of an emulsion which are subsequently sorted using fluorescence activated cell sorting FACS.

Preferably the indicator cells, the viral/effector particles and the compounds are incubated within aqueous droplets of an emulsion which are subsequently sorted using a microfluidics sorting device.

Preferably the indicator cells, the viral/effector particles and the compounds are incubated within aqueous droplets of an emulsion which is subsequently broken to enable affinity-based sorting of infected and non-infected cells.

The methods of the invention are often described in connection with inhibitors of viral entry. Clearly, the read-out used is preferably downregulation of gene(s) triggered by viral entry. However, it is important to note that said downregulation may be triggered by viral entry, or may be triggered by inhibition of early steps of the viral life cycle such as inhibition of reverse transcription, or integration into the host cell genome. The skilled addressee can easily adapt the techniques described herein to more closely connect them to such a downstream event if is it desired.

It is an advantage of the invention that use of wild type virus can be avoided. Indeed, for any given virus being studied, it is possible to eliminate all elements except the env protein of that virus using the assays of the present invention. The env protein will typically be required for pseudotyping of the effector particles being used in place of the wild type virus. This provides benefits such as safety and cost in being able to conduct the assays in low level containment facilities when avoiding wild type virus. Furthermore, it enables poorly characterized virus to be studied, since by using pseudotyped effector particles no knowledge about virally mediated downregulation of cellular proteins is required.

It is an advantage of the invention that signal amplification is enabled. Whether using direct or indirect detection of reporter gene activity, amplification can be easily introduced. For example, using direct detection antibody sandwich techniques can be used to amplify the signal, and when using these or indirect techniques involving enzymatic activity, each enzyme molecule can repeatedly turn over substrate molecules to provide more signal. This is in contrast to prior art techniques such as GFP expression where a strict 1:1 stoichiometry is inherent to the signal system.

It is an advantage of the invention that the readout is advantageously at the cell surface. In this way, substrate does not need to be able to penetrate the cell, but can be easily supplied extracellularly.

The invention is now described by way of examples, which are not intended to limit the scope of the invention but rather are intended to illustrate ways in which the invention can be worked, in which reference is made to the following figures:

### BRIEF DESCRIPTION OF THE FIGURES

FIG. 1 shows a diagram of downregulation of CD4 following viral infection.

FIG. 2 shows a diagram illustrating an application of the invention.

FIG. 3a shows a diagram showing a GFP-CD4 fusion;

FIG. 3b shows a B-lactamase-CD4 fusion according to the present invention.

FIG. 4 shows a diagram illustrating droplet-based application of the invention.

FIG. 5 shows a diagram illustrating selection of small molecule inhibitors.

FIG. 6 shows a diagram illustrating that non-potent inhibitors allow infection to proceed.

FIG. 7 shows a diagram illustrating shut down of signal upon infection.

FIG. 8 shows a graph showing expression of fusion protein.

FIG. 9 shows a graph showing downregulation of fusion protein by Nef.

FIG. 10 shows two photomicrographs of cells.

FIG. 11 shows five plots.

FIG. 12 shows a diagram of downregulation of a reporter gene following cell-entry of viral particles (A) and the tPA-PDGFR-TM fusion protein B).

FIG. 13 shows a diagram illustrating selection based on substrate conversion.

FIG. 14 shows a diagram illustrating selection based on antibody staining.

FIG. 15 shows a diagram comparing the determination of an optimal inhibitor concentration according to the invention (A) or according to the prior art (B).

FIG. 16 shows a graph showing the correlation between viral titer and readout signal.

FIG. 17 shows a graph showing the correlation between inhibitor concentration and readout signal.

FIG. 18 shows three graphs comparing a method according to the invention to determine the optimal inhibitor concentration with prior art attempts to do same.

FIG. 19 shows three plots demonstrating a selection procedure for genetically-encoded inhibitors.

FIG. 20 shows a diagram illustrating the downregulation of viral receptors upon infection.

FIG. 21 shows a diagram illustrating the downregulation of β-lac-CD4 upon infection (A) and the β-lac-CD4 fusion protein according to the present invention (B).

FIG. 22 shows a cloning diagram.

FIG. 23 shows a cloning diagram.

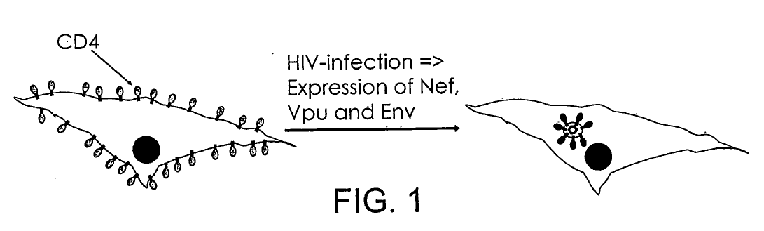

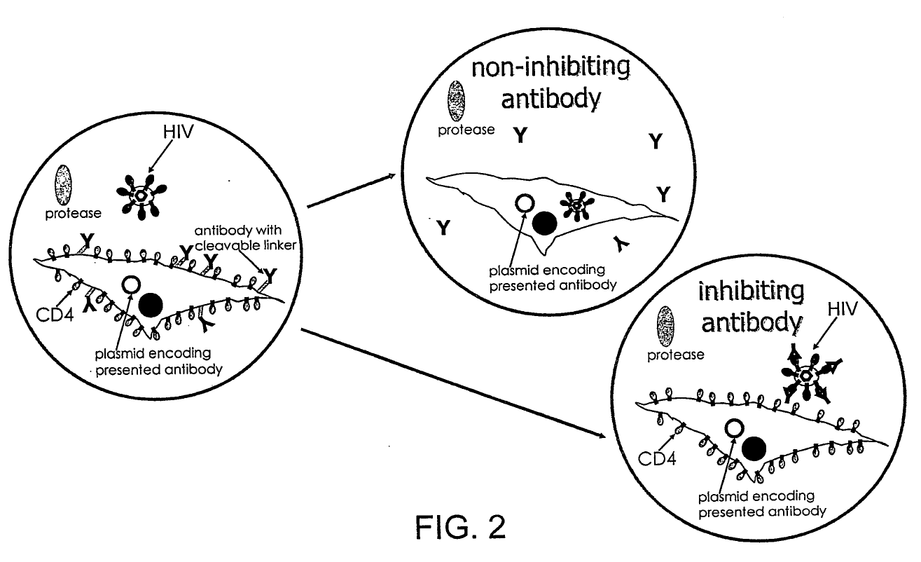

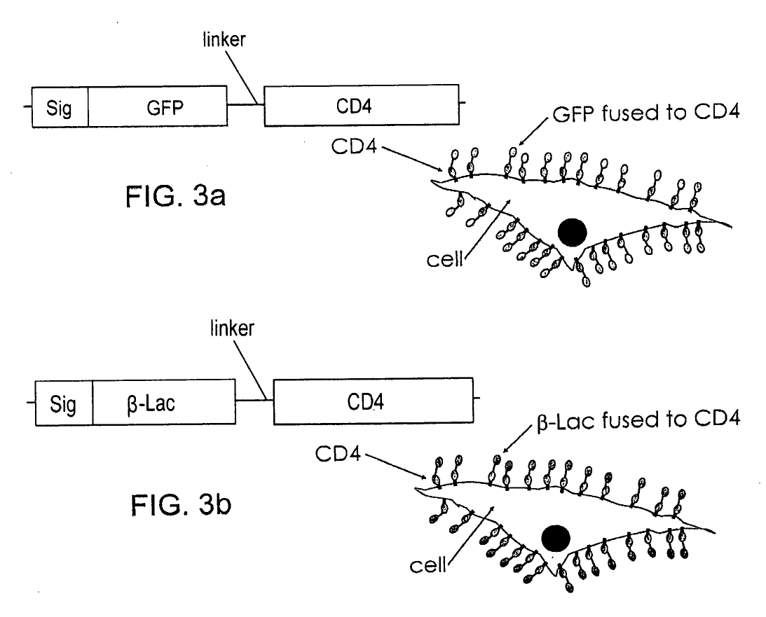

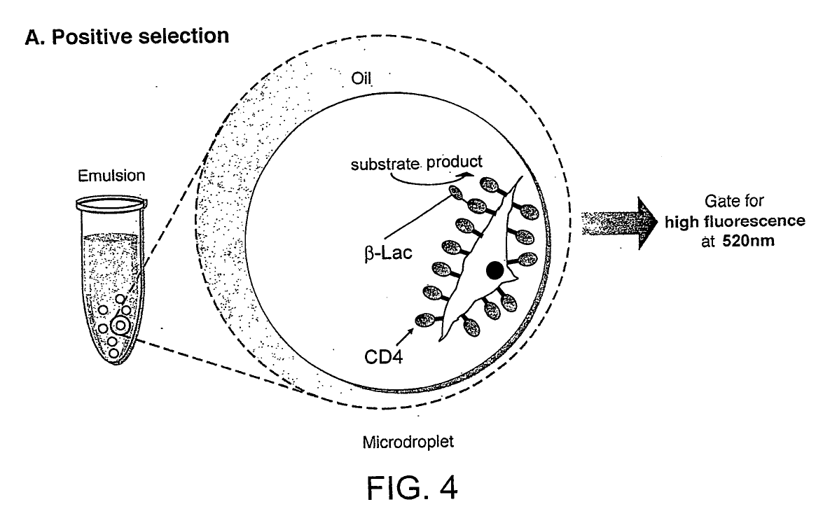

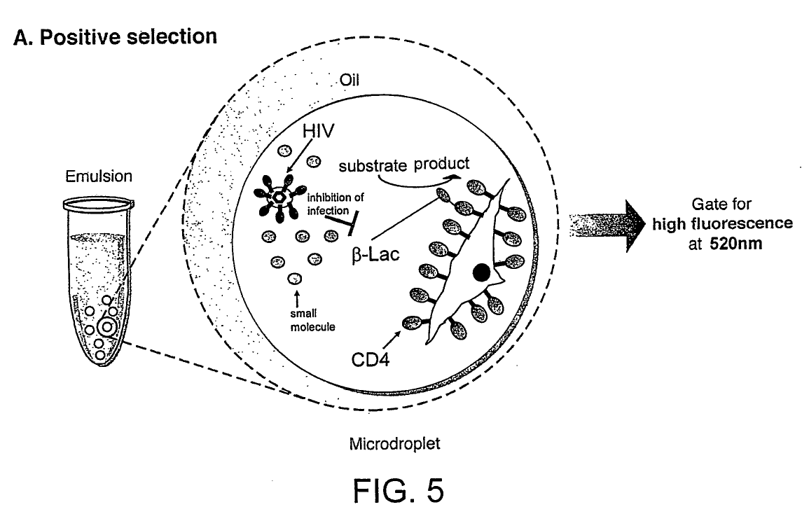

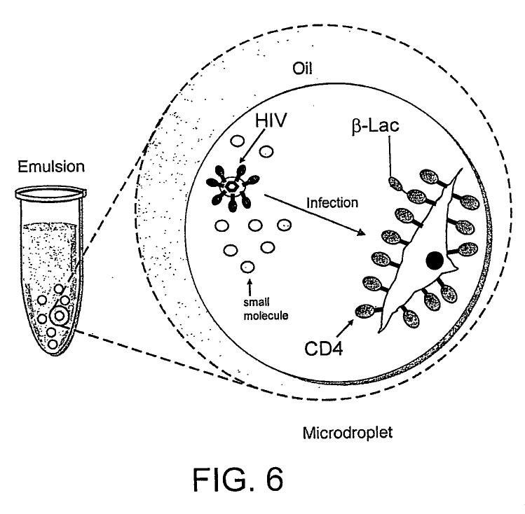

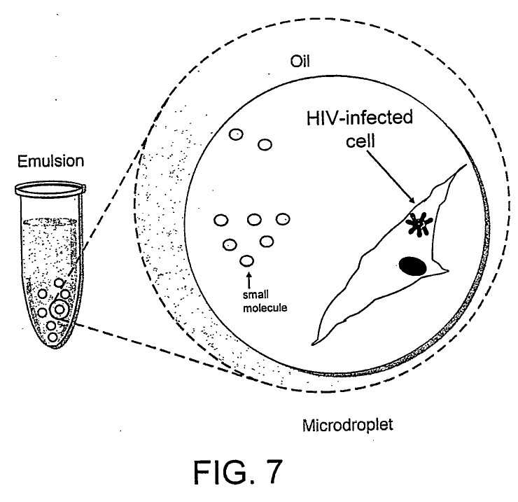

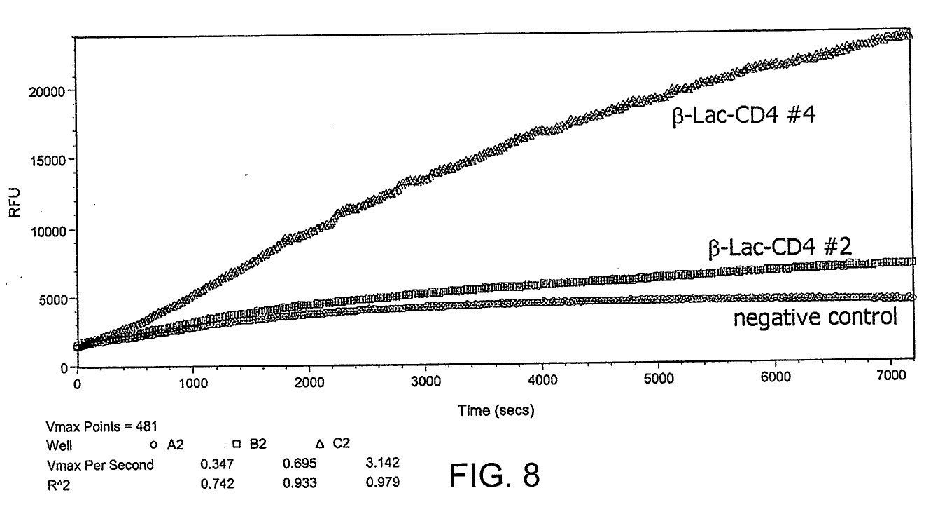

### EXAMPLES

General Technique

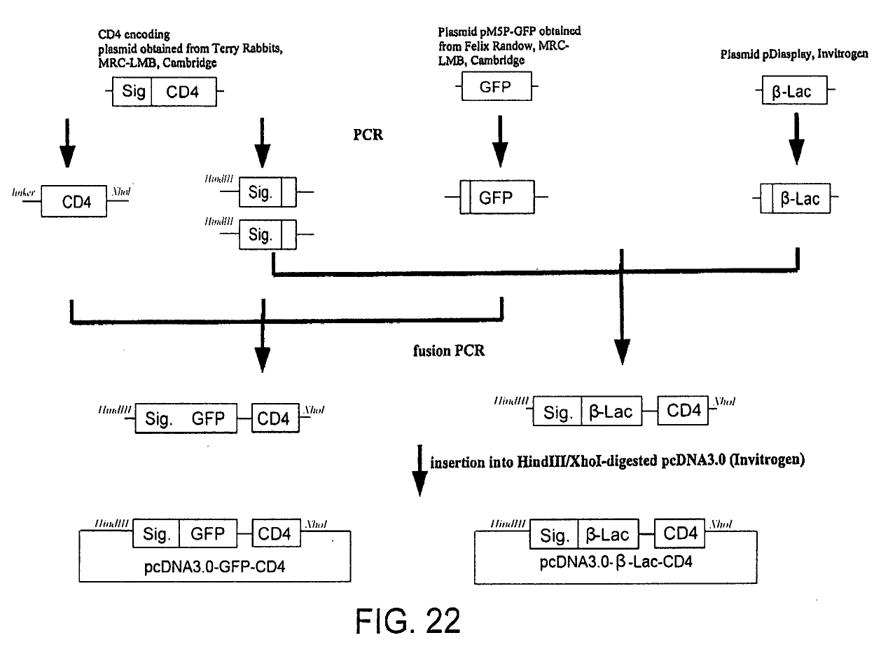

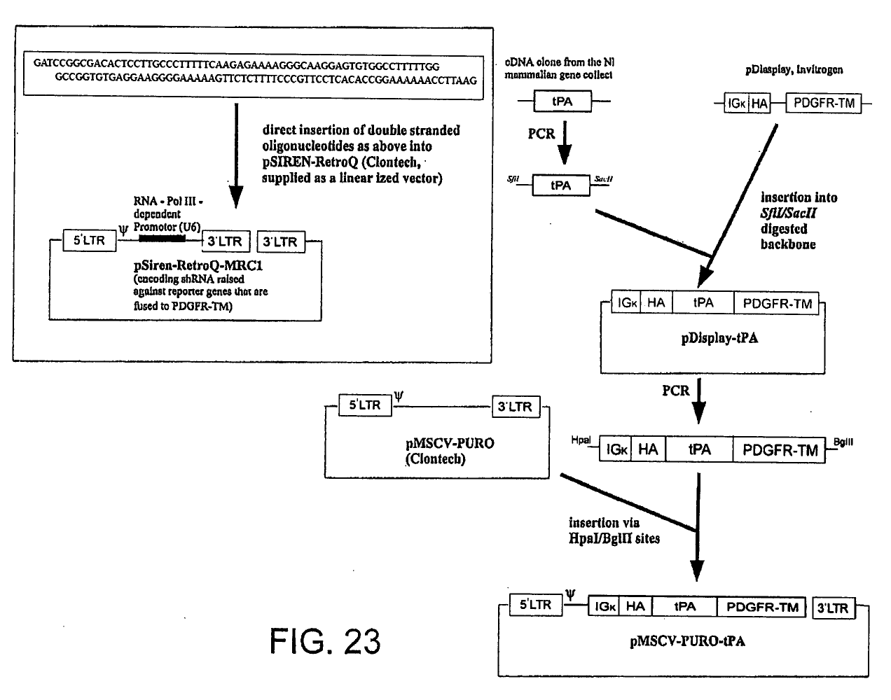

A demonstration of the principle of the assay systems of the present invention has been performed. In this case, β-lactamase was fused to the N-terminus of the CD4 receptor (C-terminal to the signal peptide), thus enabling the conversion of a non-fluorogenic substrate of β-lactamase into a fluorogenic product. The encoding plasmid was transfected into 293 EBNA T cells together with an additional plasmid encoding either the HIV gene nef (to simulate HIV infection) or a non-related control plasmid (to ensure the same amount of total DNA in both transfection samples). Two days post transfection the cells were harvested, incubated with the β-lactamase substrate and analysed within a fluorimeter. The cells expressing nef showed a more than 7-fold reduced fluorogenic signal compared to the cells which had been transfected with the control plasmid. This ratio is further increased by the generation of cells that stably express the corresponding constructs. In conclusion, this clearly demonstrates how an indicator cell line for a given virus can be generated exhibiting a fluorogenic signal unless certain viral genes are expressed ie. coupling a positive signal with a lack of infection. In the following examples, where MLV pseudotype vectors are mentioned, p-SIREN-RetroQ-MRC1 (FIG. 22/23) is the shRNA encoding vector. See sequence listing for more detail. (CD4 constructs typically use the pcDNA3.0 backbone, which is commonly available and therefore not shown in the sequence listings.)

### Example 1

### Small Molecule Screen for Inhibition of HIV Infection

In this example a compound library is screened for the ability to inhibit the infection of CD4-positive cells with the human immunodeficiency virus (HIV).

An indicator cell line is generated that stably expresses a reporter gene fused to the CD4-receptor. The reporter gene in this example is B-lac.

A wildtype CD4 receptor can be expressed additionally, if the fusion protein does not mediate cell-entry of HIV particles. In this example the CD4-B-lac fusion is functional in that the fused CD4 allows viral entry so that further expression of wildtype CD4 is not necessary.

One or more of the required coreceptors such as CXCR4, CCR5, etc. can be expressed to facilitate viral entry if required.

These indicator cells are seeded in microtiter plates and incubated with HIV-1 particles in presence of different compounds in each well.

B-lac activity is assessed by addition of substrate which is cleaved by B-lac activity into a fluorescent moiety. The action of B-lac is therefore monitored by the measurement of fluorescence at the appropriate wavelengths.

Upon infection, the reporter-CD4 fusion protein is downregulated due to the expression of the viral genes env, vpu and nef. Consequently, only cells that have not been infected with HIV will express the reporter gene. Thus, wells that exhibit a positive reporter signal contain compounds that inhibit HIV infection.

Thus, in this example, fluorescence following exposure to HIV effector particles indicates inhibition of infection and therefore indicates that the small molecule candidate(s) in those wells which exhibit fluorescence have an inhibitory effect on infection.

### Example 2

### Assay Using Psuedotyped Effector Particles

### Overview

In this example, pseudotyped effector particles are used instead of wildtype virus. These are used to deliver a shRNA to inhibit expression of a reporter gene.

Applications of the invention using pseudotyped effector particles in this manner advantageously have broader application range than those using actual viruses as effector particles, since knowledge about virus-mediated downregulation of specific proteins is not required. The shRNA is designed to a specific reporter gene, and may even be purchased from commercial suppliers, saving further effort on the part of the operator. In addition, there a major safety benefits due to the fact that the application of non-replication competent particles, such as pseudotyped particles bearing shRNA loads, decreases the containment level required to conduct the assay.

### Method

Recombinant MLV-derived pseudotype particles (effector particles) that have packaged a nucleic acid vector encoding short hairpin RNA (shRNA) raised against the reporter gene of the indicator cell line are prepared using an appropriate packaging cell line/nucleic acid shuttle system. These systems are well known in the art.

In more detail, MLV-derived particles are generated that functionally display the envelope proteins of the virus of interest, resulting in the host range tropism of that particular species (pseudotyping). Furthermore these particles are engineered to package a vector encoding shRNA raised against a reporter gene expressed in the indicator cell line. Consequently, the expression of the reporter gene is directly downregulated by the shRNA generated upon cell entry of the effector particles.

By using recombinant effector particles, the whole assay can easily be modified for different viral species. Simply by exchanging the viral envelope protein expressed in the packaging cells (and subsequently displayed on the particles), inhibitors against a variety of species can be selected. There is no need to alter the nature of the shRNA-encoding vector, nor to create a new reporter gene construct. Advantageously there is not even a requirement for species-specific indicator cells, as long as the corresponding viral receptors are expressed.

The remainder of the assay is conducted as in example 1.

### Example 3

### Selection of Candidate Inhibitors Expressed in Indicator Cells

Genetically encoded inhibitors such as antibodies or peptides are selected in a directed evolution approach. For this purpose, an indicator cell line expressing a library of inhibitors (candidate inhibitors of infection) and additionally a membrane-anchored affinity tag (reporter gene) is constructed.

Effector particles are used that have packaged a vector encoding shRNA raised against the affinity tag.

For selection, single indicator cells and effector particles are co-compartmentalised (in this example they are co-compartmentalised into aqueous droplets) and incubated to allow cell-entry of the effector particles.

In case of transduction/entry, the membrane anchored affinity tag will be downregulated by action of the shRNA.

In contrast, if a particular candidate inhibitor variant prevents cell-entry of the effector particles, the affinity tag will still be efficiently expressed.

This allows the operator to specifically select non-transduced cells. This can even be done after pooling the contents of the compartments, or optionally even after recultivating the cells.

For the selection, the cells are stained with antibodies raised against the affinity tag and applied to magnetic- or fluorescence activated cell sorting (SACS, FACS).

Once the non-transduced cells are selected, the identity of the inhibitor(s) that prevented cell-entry is determined by sequencing the nucleic acid encoding the candidate inhibitor from the recovered uninfected cells.

### Example 4

### Application to Microfluidic Handling Techniques

A non-genetically encoded small molecule library is screened for the ability to inhibit viral infection of a given species making use of automated devices.

In this example, indicator cells and effector particles (in this example the effector particles are wildtype virus particles) are incubated within compartments in the presence of single candidate inhibitor compounds.

The compartments may be the wells of microtiter plates or droplets within a microfluidics device, both of which are known in the art. The important factor is that the compound identity in each compartment is known.

Next, the reporter gene signal is determined.

This is performed in an appropriate manner with regard to the choice of reporter gene ie. fluorogenic, luminometric or chromogenic measurements are taken after addition of a corresponding substrate for the reporter gene.

Non-transduced indicator cells can be identified by the presence of good signal. Infected cells have downregulated the signal. Consequently, the compound(s) present in the wells corresponding to good signal are identified as having an inhibitory effect on cell entry/viral infection.

### Example 5

### Correlation of Effector Particle Entry with Readout Signal

Different amounts of effector particles have been incubated with the indicator cells. Subsequently, fluorescence assays based on the conversion of the non-fluorogenic plasmin substrate into a fluorogenic product have been performed. Indicator cells that have not been incubated with effector particles exhibited an up to 10-fold higher fluorescence signal than indicator cells that have been incubated with highly concentrated effector particles. Furthermore, dilution of the effector particles prior to incubation with the indicator cells resulted in an intermediate intensity of the fluorescence signal. This clearly shows that the number of transduction events directly correlates with the readout signal.

### Example 6

### Demonstration of Assay Inhibition Signal Using AZT Inhibitor

To check the influence of a potent viral inhibitor on the readout signal, indicator cells and effector cells have been incubated in presence or absence of the reverse transcriptase inhibitor AZT. In absence of AZT the indicator cells showed the lowest fluorescence signal, whereas increasing concentrations of the inhibitor resulted in increased signal intensities.

A further experiment was performed to show that adverse side effects of the inhibitor on the indicator cells result in a decreased readout signal. For that purpose effector particles and indicator cells were incubated in presence of AZT concentrations of up to 1 mM (cytotoxic inhibitor concentrations). Using the fluorescence-based readout, the signal intensity increased up to a certain optimal concentration of AZT (10 um), whereas even higher concentrations of the inhibitor resulted in decreased readout signals. In good agreement with that, the fluorescence signal of indicator cells that were treated with AZT in absence of any particles showed decreasing fluorescence intensities with increasing inhibitor concentrations (due to the cytotoxic effects of AZT). Subsequently, the experiment was repeated with a different type of particles.

As a comparison with prior art expression induction viral inhibition assays using GFP induction upon infection, MLV(VSV-G Env) particles were generated that transduce a gfp-gene into the indicator cell line upon effector particle entry. As expected, the lowest fluorescence signal (within this kind of assay the phenotype for the optimal inhibitor concentration) was obtained when using the highest inhibitor concentration, far within the cytotoxic range. This clearly shows that prior art expression induction based assays are potentially biased in favour of cytotoxic inhibitors. In contrast, the present invention advantageously enables identification of cytotoxic inhibitors or cytotoxic concentrations of inhibitors.

### Example 7

### Expression Library Screening

The invention can also be used to select genetically encoded inhibitors of viral cell-entry (or inhibitors of the subsequent reverse transcription step) in a directed evolution approach. For this purpose, indicator cells expressing a library of potential inhibitors (e.g. intracellular peptides) are incubated with the effector particles. After the transduction step, non-transduced indicator cells can specifically be identified/selected due to the level of surface-displayed tPA-HA, which will decrease upon cell-entry of the effector particles. These non-transduced indicator cells will have either resisted effector particle entry, or more probably when using intracellular peptide libraries may have resisted transduction due to the expression of a downstream inhibitor such as a potent reverse transcriptase inhibitor variant.

Using antibodies raised against the tPA-HA, fluorescence activated cell sorting (FACS) allows the screening of millions of indicator cells for the transduction event. After specific selection of non-transduced indicator cells, the encoded inhibitor variants can be recovered by PCR on DNA from the selected indicator cells.

As a demonstration of this use of the invention, indicator cells have been incubated with effector particles in presence or absence of AZT (as a substitute for a genetically-encoded inhibitor) and stained subsequently with \acute{α}-HA antibodies. While the sample containing 25 uM AZT and an untransduced control sample show just one (fluorescence positive) population, the sample without the inhibitor shows two populations with the majority of indicator cells being fluorescent negative. This clearly shows that the invention allows specific selection for non-transduced cells potentially expressing potent inhibitors of viral infection (effector particle entry).

### Example 8

### Downregulation of Cell Surface-Displayed Reporter Genes

A demonstration of the principle of the assay systems of the present invention has been performed. In the example, the cell surface displayed reporter gene is a recombinant tPA fusion.

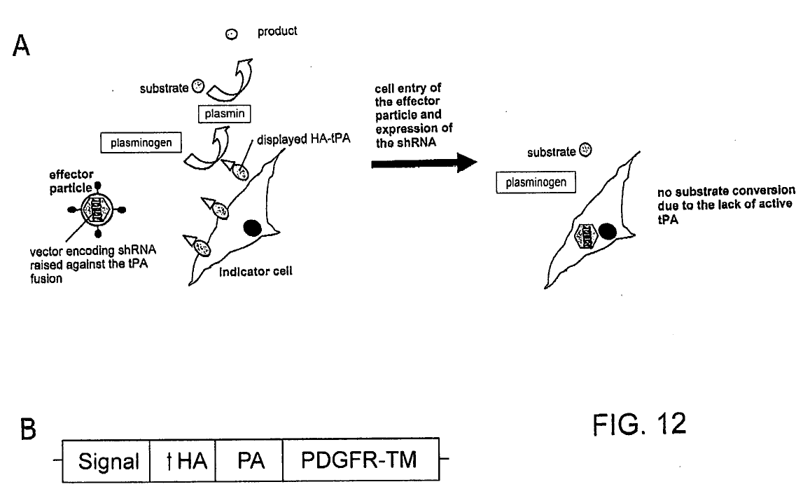

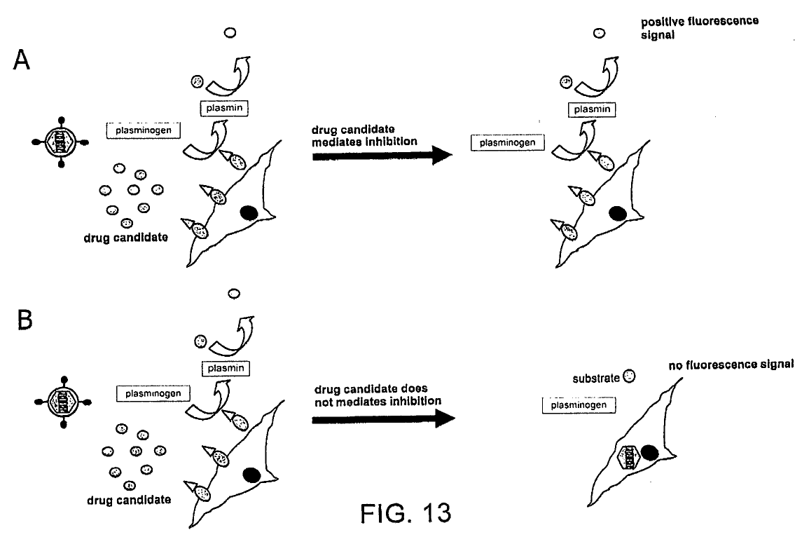

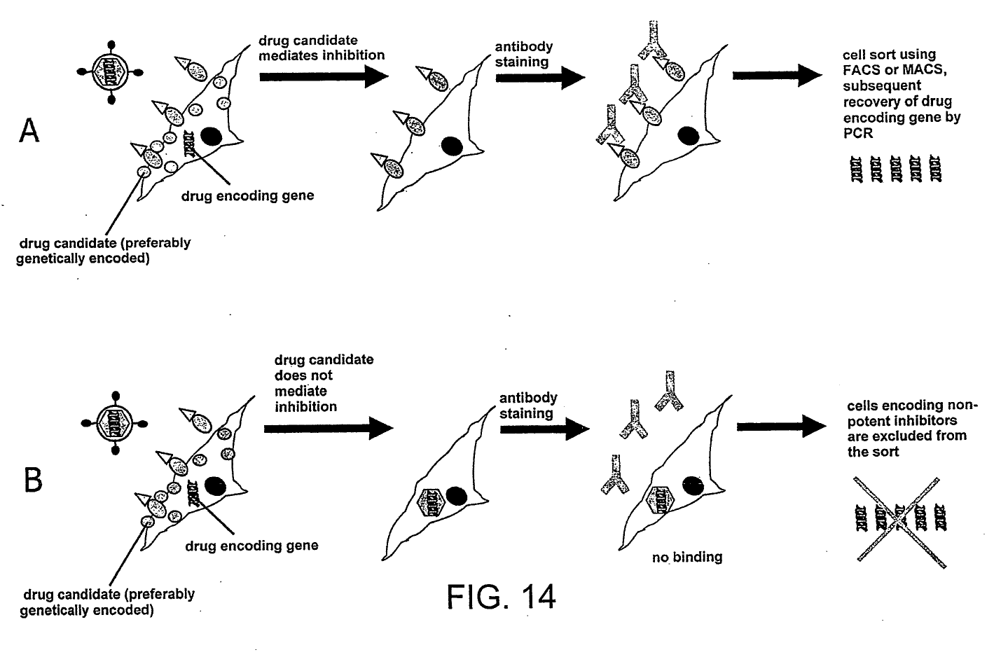

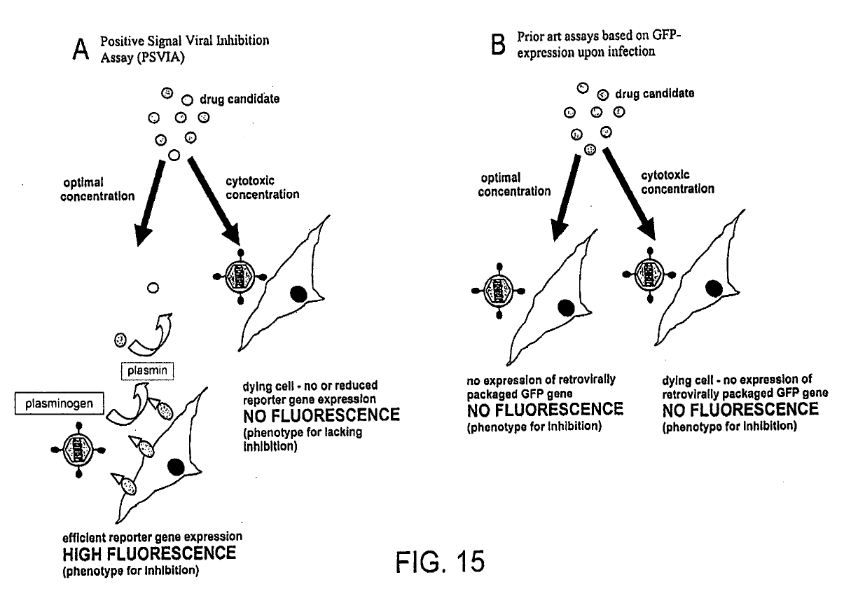

An HA-tagged version of human tPA is fused to the N-terminus of PDGFR-TM (C-terminal to an IG-K chain signal sequence; FIG. 12B). The resulting fusion protein is able to convert plasminogen into plasmin which then converts a non-fluorogenic substrate (in this example HDLVK-Amc) into a fluorogenic product. An indicator cell line stably expressing this reporter gene construct is generated by retroviral transduction of HEK293T cells with the corresponding gene. As effector particles, MLV(VSV-G Env) pseudotype particles are generated. These particles have packaged a vector encoding shRNA against the tPA-HA. Upon cell entry of the effector particles, the shRNA is expressed in the indicator cells resulting in downregulation of the tPA-HA.

Signal Correlates with Effector Particle Entry

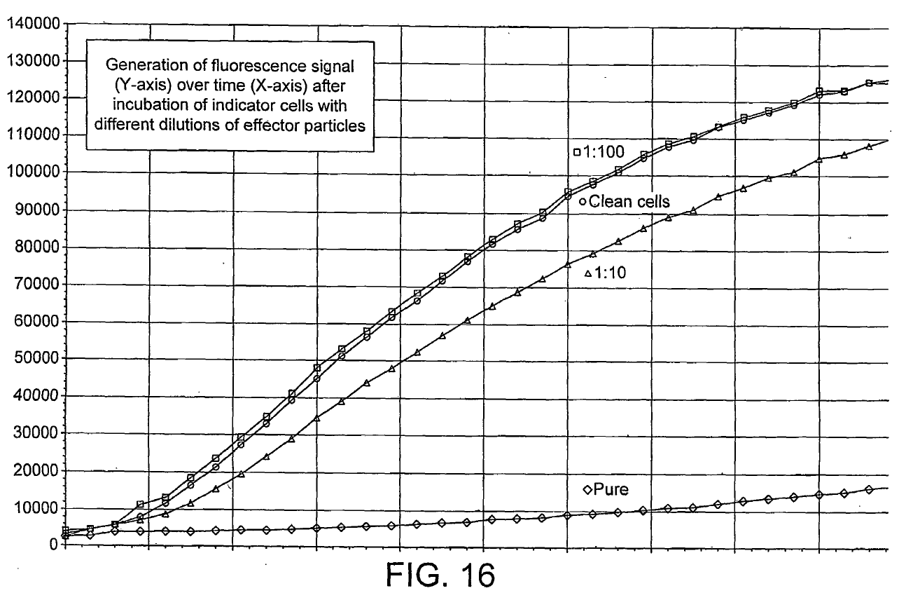

Different amounts of effector particles have been incubated with the indicator cells (FIG. 16). Subsequently, fluorescence assays based on the conversion of the non-fluorogenic plasmin substrate into a fluorogenic product are performed. Indicator cells that had not been incubated with effector particles exhibited an up to 10-fold higher fluorescence signal than indicator cells that had been incubated with concentrated effector particles. Furthermore, dilution of the effector particles prior to incubation with the indicator cells resulted in an intermediate intensity of the fluorescence signal. This clearly shows that the number of transduction events directly correlates with the readout signal.

### Inhibition of Infection Maintains Signal

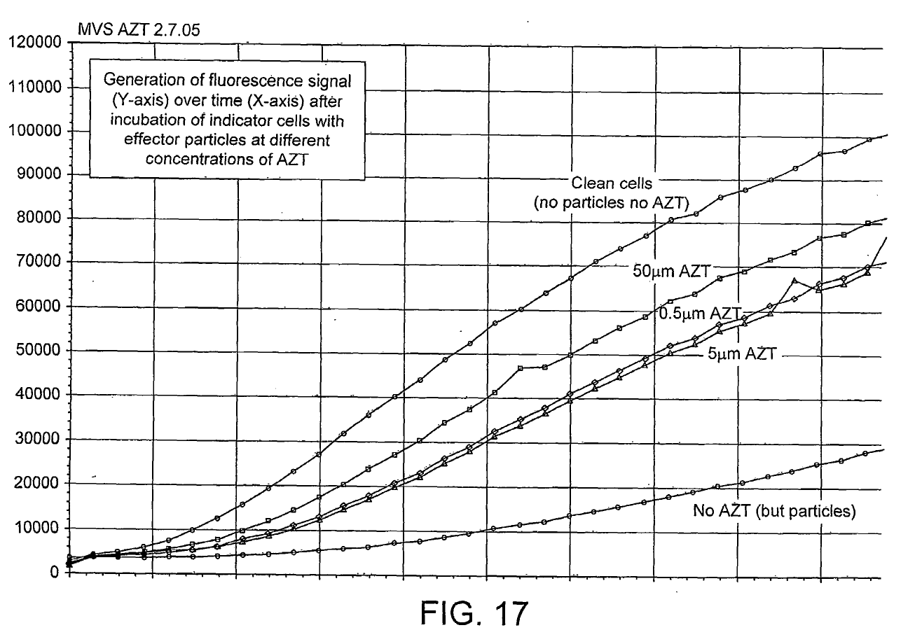

To show the influence of a potent viral inhibitor on the readout signal, indicator cells and effector particles have been incubated in presence or absence of the reverse transcriptase inhibitor AZT (FIG. 17). In the absence of AZT the indicator cells showed the lowest fluorescence signal, whereas increasing concentrations (0.5-50 μM) of the inhibitor resulted in increased signal intensities.

### Cytotoxicity Reduces Signal

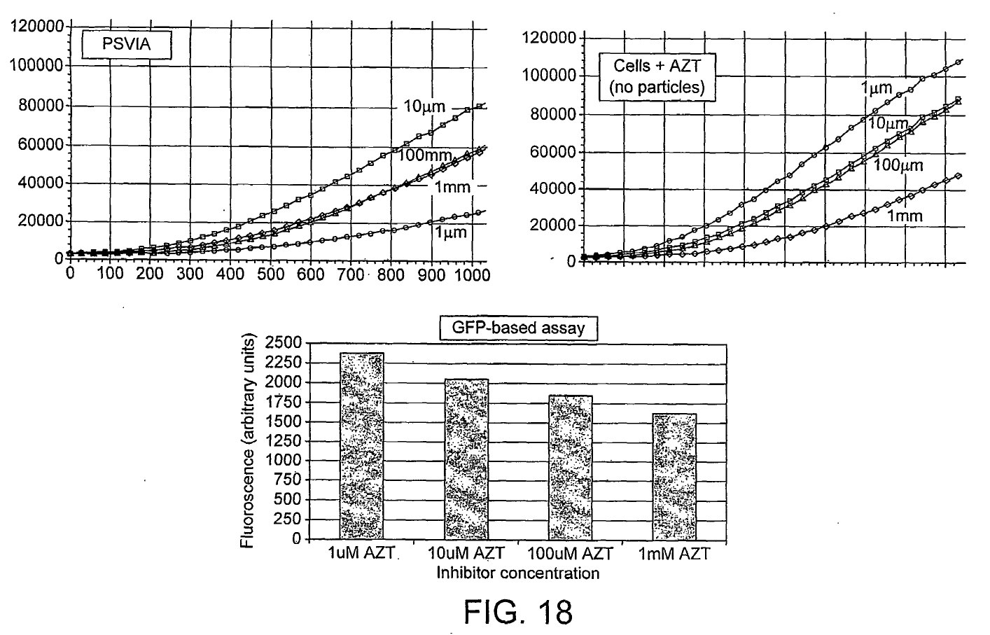

A further experiment (FIG. 18) was performed to show that adverse side effects of the inhibitor on the indicator cells result in a decreased readout signal. For that purpose effector particles and indicator cells are incubated in presence of AZT concentrations of up to 1 mM (cytotoxic inhibitor concentrations). Using the fluorescence-based readout, the signal intensity increased up to a certain optimal concentration of AZT (10 μm), whereas even higher concentrations of the inhibitor resulted in decreased readout signals. In good agreement with this, the fluorescence signal of indicator cells that were treated with AZT in absence of any particles showed decreasing fluorescence intensities with increasing inhibitor concentrations (due to the cytotoxic effects of AZT).

### Comparative Example

A comparative experiment was performed as above with a different type of particle. As a model for prior art gfp-based viral inhibition assays, MLV(VSV-G Env) particles were generated that transduce a gfp-gene into the indicator cell line upon effector particle entry, so that signal correlates with infection (rather than signal correlating with inhibition of infection as in the present invention). As expected, the lowest fluorescence signal (which within this kind of assay is regarded as the phenotype for the optimal inhibitor concentration) was obtained when using the highest inhibitor concentration, well within the cytotoxic range. This clearly shows that viral inhibition assays coupling infection to expression (such as gfp-based assays of the prior art) are apparently biased in favour of cytotoxic inhibitors. By contrast, the assays of the present invention allow identification of cytotoxic inhibitors (or cytotoxic concentrations thereof).

### Example 9

### Screening of Expression Libraries

The assays of the invention can also be used to select genetically encoded inhibitors of viral entry (effector particle entry) in a directed evolution approach. For this purpose, indicator cells expressing a library of potential inhibitors (e.g. intracellular peptides) are incubated with the effector, particles. After the transduction step, non-transduced indicator cells (probably due to the expression of a potent inhibitor variant) can specifically be identified/selected due to the level of surface-displayed tPA-HA, which decreases upon cell-entry of the effector particles.

In order to perform selection, using antibodies raised against the tPA-HA, standard cell sorting techniques (such as FACS and MACS) allow the screening of millions of indicator cells for the transduction event. After specific selection of non-transduced indicator cells, the encoded inhibitor variants can be recovered by PCR on cellular (library) DNA.

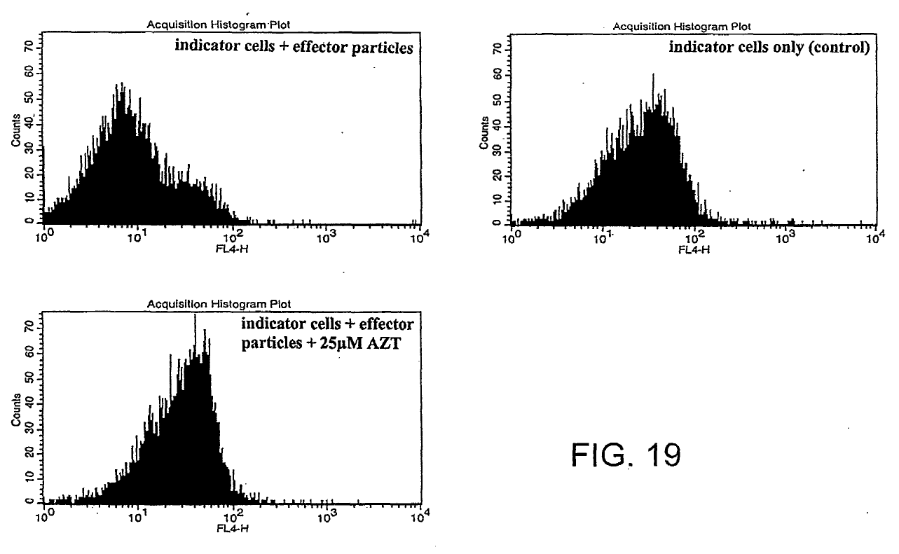

In order to demonstrate this application of the invention, indicator cells have been incubated with effector particles in presence or absence of AZT (as a substitute for a genetically-encoded inhibitor) and stained subsequently with \acute{α}-HA antibodies (FIG. 19). While the sample containing 25 uM AZT and an untransduced control sample show just one (fluorescence positive) population, the sample without the inhibitor shows two populations with the majority of indicator cells being fluorescent negative. This clearly shows that the assay of the invention allows to specifically select for non-transduced cells potentially expressing potent inhibitors of viral infection.

Clearly this assay can be easily used to detect inhibitors of downstream events following infection such as the reverse transcription step. Expressed internal peptides are particularly suited to this assay since the reverse transcriptase step is internal to the cell.

### Example 10

### Downregulation of Reporter Genes Fused to Receptors

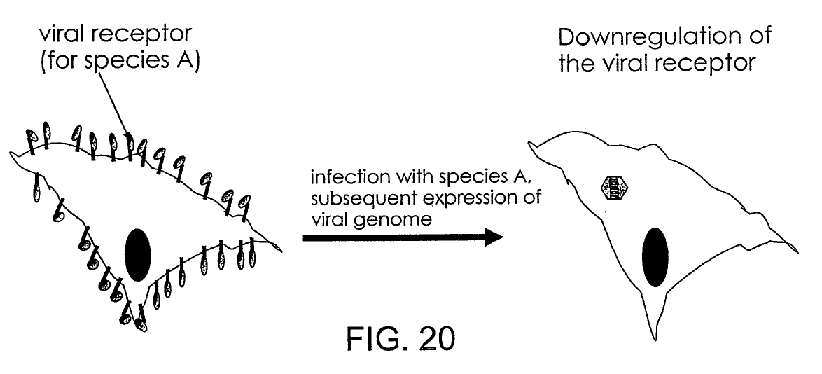

Instead of fusing the reporter gene to a transmembrane domain (such as the non-viral PDGF-TM domain), the reporter can be fused to a cellular protein known to be downregulated upon viral infection (e.g. a viral receptor such as CD4; FIG. 20).

In this example, the downregulation of CD4 upon expression of HIV nef has been used to demonstrate this strategy.

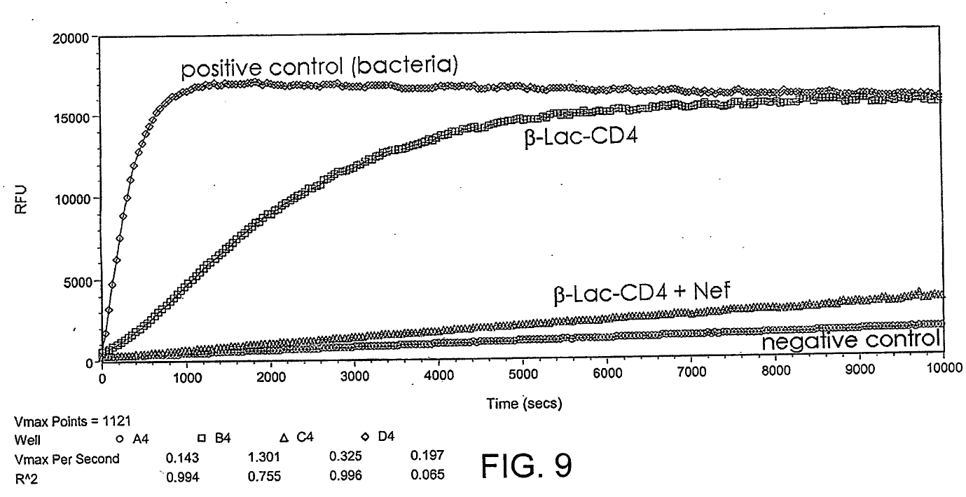

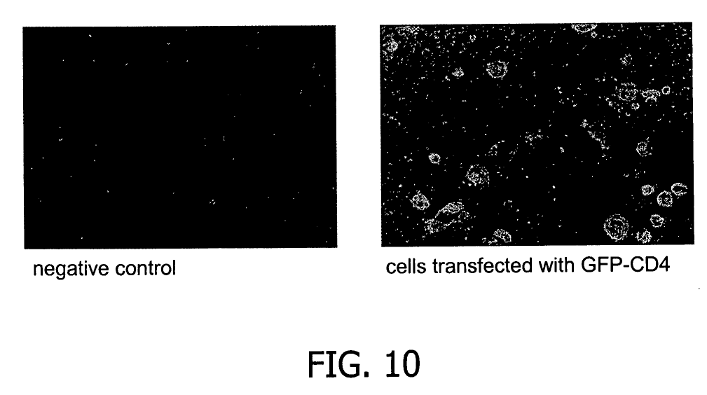

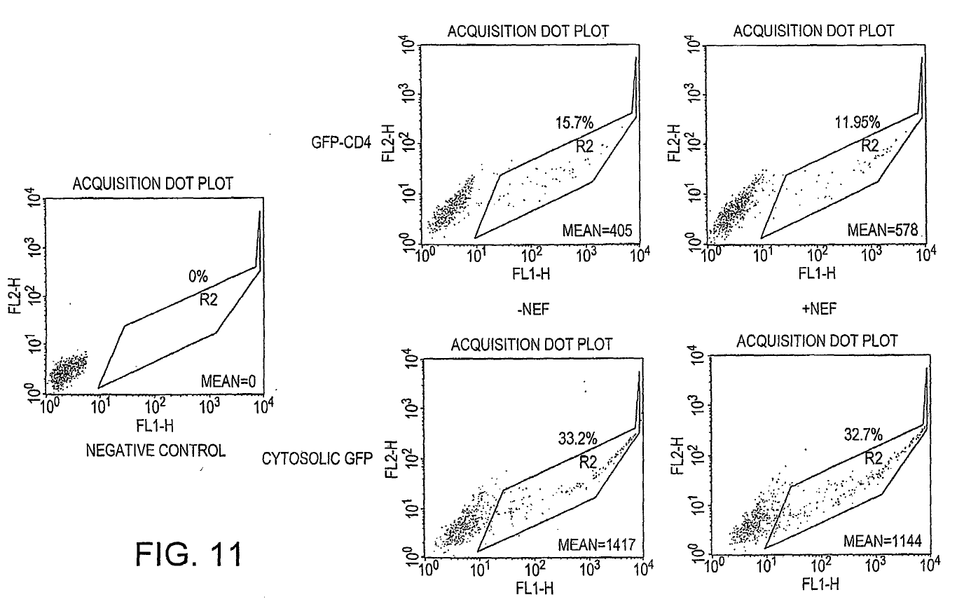

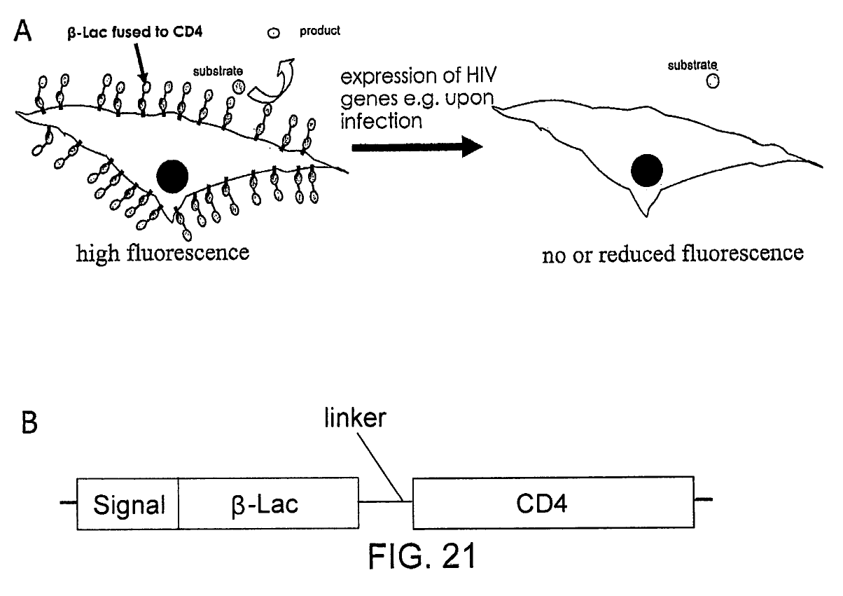

In this example, β-lactamase was fused to the N-terminus of the CD4 receptor (C-terminal to the signal peptide; FIGS. 21A and 21B), thus enabling the conversion of a non-fluorogenic substrate of β-lactamase into a fluorogenic product. The encoding plasmid was transfected into HEK293 EBNA T cells together with an additional plasmid encoding either the HIV gene nef (to simulate HIV infection) or a non-related control plasmid (to ensure equal amounts of total DNA in both transfection samples). Two days post transfection the cells were harvested, incubated with the β-lactamase substrate and analysed within a fluorimeter. The cells expressing nef showed a more than 7-fold reduced fluorescence signal compared to the cells which had been transfected with the control plasmid (FIG. 9). This ratio is further increased by the generation of cells that stably express the corresponding constructs. In conclusion, this clearly demonstrates how a reporter gene fused to a viral receptor generates a positive fluorescence signal unless certain viral genes are expressed (simulating viral entry) causing downregulation of the corresponding reporter protein. Hence, coupling a positive signal to a lack of infection is achieved.

In an application where a reporter-viral receptor fusion protein does not support cell-entry of the corresponding viral particles (effector particles), a wildtype viral receptor can also be expressed in the indicator cells in order to support effector particle entry.

### Example 11

### Small Molecule Screen for Inhibition of Infection

In this example a compound library is screened for the ability to inhibit the infection of cells permissive for a viral species of interest.

An indicator cell line is generated by that stably expresses a reporter gene either fused to a non-viral transmembrane domain or a viral receptor. The indicator cells in this example are made from hepatocytes. The reporter gene in this example is HA-tPA fused to PDGFR-TM. This construct and all receptors required for cell-entry of the viral species of interest are expressed in the indicator cell line.

These indicator cells are seeded in microtitre plates and incubated with effector particles having packaged a vector encoding shRNA against the reporter gene construct (here: HA-tPA-PDGFR-TM) and pseudotyped with the envelope protein of interest. The effector particles in this example comprise the envelope glycoproteins of Hepatitis C Virus (E1 and E2), displayed on MLV particles.

tPA activity is assessed by addition of plasminogen and a substrate which is cleaved by plasmin (upon tPA-mediated conversion of plasminogen into plasmin) activity into a fluorescent moiety. The action of tPA is therefore monitored by the measurement of fluorescence at the appropriate wavelengths.

Upon cell-entry/transduction, the reporter fusion protein is downregulated due to the expression of the shRNA-encoding vector. Consequently, only cells that have not been transduced by the effector particles will express the reporter gene. Thus, wells that exhibit a positive reporter signal contain compounds that inhibit infection of the viral species of interest (the species from which the envelope protein was derived).

Thus, in this example, fluorescence following exposure to effector particles indicates inhibition of infection and therefore indicates that the small molecule candidate(s) in those wells which exhibit fluorescence have an inhibitory effect on infection.

1. A method for identifying inhibitors of viral entry comprising
(i) providing an indicator cell wherein said cell expresses a reporter gene and wherein said cell is capable of supporting entry by an effector particle
(ii) providing a candidate inhibitor of viral entry;
(iii) co-compartmentalizing said candidate inhibitor and said indicator cell;
(iv) contacting said indicator cell with an effector particle;
(v) incubating to allow any effector particle entry to take place; and
(vi) assaying said indicator cell for report gene activity, wherein detection of reporter gene activity identifies the candidate inhibitor as an inhibitor of viral entry.

2. A method according to claim 1 wherein the reporter gene product is directed to the cell surface.

3. A method according to claim 2 wherein that part of the reporter gene product which mediates detection is extra cellular.

4. A method according to claim 1 wherein said reporter gene encodes β-lactamase or tissue plasminogen activator (tPA).

5. A method according to claim 1 wherein said reporter gene is fused to a gene known to be down regulated upon viral entry.

6. A method according to claim 5 wherein said gene known to be down regulated on viral entry is CD4.

7. A method according to claim 1 where said reporter gene comprises a CD4-β-lactamase fusion.

8. A method according to claim 1 wherein said reporter gene is under the control of a promoter which is known to be down regulated on viral entry.

9. A method according to claim 1 where said effector particle comprises nucleic acid encoding elements capable of inhibiting expression of the reporter gene.

10. A method according to claim 9 wherein said effector particle comprises nucleic acid encoding shRNA capable of inhibiting expression of the reporter gene.

11. A method according to claim 1 wherein said effector particle comprises a virus.

12. A method according to claim 11 wherein said virus is a wild-type virus.

13. A method according to claim 11 wherein said virus is a recombinant virus.

14. a method according to claim 13 wherein said virus is a pseudotyped virus.

15. A method according to claim 1 wherein said co-compartmentalization is by forming one or more aqueous droplets comprising both the candidate inhibitor and the indicator cell.

16. A method according to claim 15 wherein said aqueous droplets are part of water-in-oil emulsion.

17. A method according to claim 15 or 16 wherein said aqueous droplets are part of a water-in-oil water emulsion.

18. A method according to claim 1 wherein said candidate inhibitor is produced by the indicator cell.

19. A method according to claim 1 wherein the reporter gene encodes an enzyme or an active fragment thereof, and wherein detection of reporter gene activity comprises
(i) containing said indicator cell with a substrate for said enzyme;
(ii) incubating to allow said enzyme to act on said substrate; and
(iii) detecting the presence of enzymatic product, presence of the product indicating reporter gene activity.

20. A method according to claim 1 wherein detection of reporter gene activity comprises detection of reporter gene expression by
(i) contacting said indicator cell with an antibody capable of reacting with said reporter gene product;
(ii) incubating to allow binding of said antibody to said reporter gene product; and
(iii) detecting the presence of bound antibody on said indicator cell, presence of the antibody indicating reporter gene expression.

21. A method according to claim 4 wherein said reporter gene encodes tissue plasminogen activator (tPA).

22. A nucleic acid comprising the nucleotide sequence of SEQ ID NO:1, SEQ ID No:2, SEQ ID NO:3 or SEQ ID NO:4.

23. A polypeptide comprising the amino acid sequence encoded by the nucleotide sequence of SEQ ID NO:1, SEQ ID NO:2, SEQ ID NO:3 or SEQ ID NO:4.
